(19) United States
(12) Patent Application Publication
(10) Pub. No.: US 2021/0400971 A1
(43) Pub. Date: Dec. 30, 2021

(54) SAFENING COMPOSITIONS COMPRISING A PHENOXY HERBICIDE AND CLOQUINTOCET FOR CEREAL CROPS AND METHODS THEREOF

(71) Applicant: DOW AGROSCIENCES LLC, Indianapolis, IN (US)

(72) Inventors: Rory Frank Degenhardt, Edmonton (CA); Andrew Macrae, Winnipeg (CA)

(73) Assignee: DOW AGROSCIENCES LLC, Indianapolis, IN (US)

(21) Appl. No.: 17/290,235

(22) PCT Filed: Oct. 31, 2019
(86) PCT No.: PCT/US2019/059015

§ 371 Date: Apr. 29, 2021

Related U.S. Application Data
(60) Provisional application No. 62/755,111, filed on Nov. 2, 2018.

(51) Int. Cl. A01N 43/42; A01N 25/32

(52) U.S. Cl. CPC A01N 43/42; A01N 25/32

(57) ABSTRACT

Disclosed herein includes a safening composition comprising an herbicidally effective amount of (a) at least one phenoxy herbicide, or an agriculturally acceptable salts, esters, and amides thereof; (b) at least one safener from quinolinyloxyacetate family of chemicals, or agriculturally acceptable salts, esters and amides thereof, wherein the weight ratio of the at least one phenoxy herbicide to cloquintocet is from about 500:1 to about 1:1. Herbicidal injury caused by a phenoxy herbicide in wheat and barley is reduced with the use of low rates of cloquintocet.

### CROSS-REFERENCE TO RELATED APPLICATIONS

This application is an International Application, which claims priority to U.S. Provisional Application No. 62/755,111, filed Nov. 2, 2018, titled SAFENING COMPOSITIONS COMPRISING A PHENOXY HERBICIDE AND CLOQUINTOCET FOR CEREAL CROPS AND METHODS THEREOF, the entire contents of which are incorporated herein by reference.

### FIELD

Provided herein includes safening compositions comprising at least one phenoxy herbicide and cloquintocet, or agriculturally acceptable salts, esters and amides thereof. Provided herein are also methods for protecting crops from herbicidal injury caused by a phenoxy herbicide or compositions comprising at least one phenoxy herbicide disclosed herein.

### BACKGROUND

When agrochemicals, such as plant protection agents and especially herbicides, are used, the cultivated plants may be damaged to a certain degree, depending on factors such as the dose of agrochemicals and their method of application, the species of cultivated plant, the nature of the soil and climatic conditions, for example, length of time of exposure to light, temperature and amounts of precipitation. Thus, it is known that cultivated plants which are to be protected from the adverse effect of undesirable plant growth may be damaged to a certain degree when an effective dose of herbicide is used. Various substances which are capable of specifically preventing the adverse effect of an herbicide on the cultivated plants, i.e. of protecting the cultivated plants without at the same time noticeably influencing the herbicidal action on weeds to be combated, have been proposed to solve this problem. However, it has been found that the antidotes proposed frequently have only a narrow field of use, i.e., a particular antidote is frequently suitable only for use with individual species of cultivated plants and/or for protecting the cultivated plants from individual herbicidal substances or classes of substances. It has also been found that the antidotes proposed frequently are used at rates higher than the rates of the individual herbicidal substance.

Phenoxy herbicides including, but are not limited to, 2,4-D, have been used for selective broadleaf weed control in wheat since the late 1940s; however, they can cause harmful effects on wheat yields if applied improperly. For example, winter wheat is susceptible to phenoxy herbicide injury from emergence to the 4-leaf stage and from jointing to the soft dough stage of growth. See, e.g., Whitesides, R. E., Identification of growth stages in winter wheat and response to broadleaf weed herbicides. Proc. West. Soc. Weed Sci. 36: 123-124. Phenoxy herbicide application at these stages can cause various harmful effects including, but are not limited to, reduced plant height, developmental deformities, delayed maturity, and reduced yields.

Many studies have reported results from the application of 2,4-D to cereal crops. Some studies have reported that spike abnormalities were evident in greatest numbers following treatment with 2,4-D when the plants had 3-5 leaves and the height of plants were about 5-8 inches. Godbout E. applied an amine and an ester of 2,4-D to wheat, oats, and barley at the three-leaf, five-leaf, and early boot stages and found that the damage was greatest where the application was made at the three-leaf stage. See, e.g., Godbout E. Effect of 2,4-D on oats at different stages of growth. Res. Rpt. Sixth Ann. North Central Weed Control Conf. Sioux Falls, S. Dak., 108. (1945). Many studies also reported damages to wheat, oats and barley in early growth stages, variously described as seedling, two-leaf, and three-leaf, especially where an ester of 2,4-D was used. Duane A. M. et al. further found that 2,4-D amine and 2,4-D ester reduced wheat yield 35% and 39%, respectively, when applied at Stage 13; and 2,4-D amine and 2,4-D ester reduced wheat yield 15% and 16%, respectively, when applied at Stage 44. Typically, it is recommended that the phenoxy herbicides including 2,4-D be applied from a well advanced seedling stage (Stage 14, generally 6 or more inches tall) up to flag leaf emergence (Stage 37). See, e.g., Commercial Catalog for 2,4-D Ester 700, Nufarm Agriculture Inc. Applications prior to Stage 14 (four-leaf stage) or after Stage 37 (including boot stage) based on BBCH Growth Stages are not recommended. See, for example, Uwe Meier, Growth stages of mono- and dicotyledonous plants, BBCH Monograph, 2nd Edition, 2001.

Thus, there remains a need for compositions and methods that are effective in ameliorating such herbicidal injury.

### SUMMARY

A first embodiment provided herein includes a safening composition comprising an herbicidally effective amount of (a) at least one phenoxy herbicide, or an agriculturally acceptable salts, esters, and amides thereof; (b) at least one safener from quinolinyloxyacetate family of chemicals, or agriculturally acceptable salts, esters and amides thereof, wherein the weight ratio of the at least one phenoxy herbicide to cloquintocet is from about 500:1 to about 1:1.

A second embodiment includes the composition according to the first embodiment, wherein the (a) at least one phenoxy herbicide, or an agriculturally acceptable salts, esters, and amides thereof comprises 2,4-D, 2,4-D EHE, 2,4-D DMA, 2,4-D choline, and/or 2,4-DB.

A third embodiment includes the composition according to the first or the second embodiments, wherein the (b) at least one safener from quinolinyloxyacetate family of chemicals comprises cloquintocet, or an agriculturally acceptable salts, esters, and amides thereof.

A fourth embodiment includes the composition according to any one of the first to the third embodiments, wherein the at least one safener is cloquintocet acid, wherein the weight ratio of the at least one phenoxy herbicide, or an agriculturally acceptable salts, esters, and amides thereof to cloquintocet acid given in units of gae/ha (grams acid equivalent per hectare) to gai/ha (grams active ingredient per hectare) or gae/ha to gae/ha comprises from about 500:1 to about 1:1, from about 400:1 to about 1:1, from about 300:1 to about 1:1, from about 200:1 to about 1:1, from about 100:1 to about 1:1, from about 99:1 to about 1:1, from about 98:1 to about 1:1, from about 95:1 to about 1:1, from about 90:1 to about 1:1, from about 80:1 to about 1:1, from about 70:1 to about 1:1, from about 60:1 to about 1:1, from about 50:1 to about 1:1, from about 40:1 to about 1:1, from about 30:1 to about 1:1, from about 20:1 to about 1:1, from about 11:1 to about 1:1, from about 10:1 to about 1:1, from about 9:1 to about 1:1, from about 8:1 to about 1:1, from about 7:1 to about 1:1, from about 6:1 to about 1:1, from about 5:1 to about 1:1, from about 4:1 to about 1:1, from about 3:1 to about 1:1, from about 2:1 to about 1:1, from about 1:1 to about 1:1, about 300:1, about 200:1, about 99:1, about 98.5:1, about 50:1, about 22:1, about 20:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, or any combination thereof.

A fifth embodiment includes the compositions according to any one of the first to the fourth embodiments, wherein the at least one safener is cloquintocet acid, wherein the weight ratio of the at least one phenoxy herbicide, or an agriculturally acceptable salts, esters, and amides thereof to cloquintocet acid given in units of gae/ha (grams acid equivalent per hectare) to gai/ha (grams active ingredient per hectare) or gae/ha to gae/ha comprises from about 500:1 to about 10:1, from about 400:1 to about 10:1, from about 300:1 to about 10:1, from about 200:1 to about 10:1, from about 100:1 to about 10:1, from about 99:1 to about 10:1, from about 98:1 to about 10:1, from about 95:1 to about 10:1, from about 90:1 to about 10:1, from about 80:1 to about 10:1, from about 70:1 to about 10:1, from about 60:1 to about 10:1, from about 50:1 to about 10:1, from about 40:1 to about 10:1, from about 30:1 to about 10:1, from about 20:1 to about 10:1, from about 11:1 to about 10:1, about 500:1 to about 20:1, from about 400:1 to about 20:1, from about 300:1 to about 20:1, from about 200:1 to about 20:1, from about 100:1 to about 20:1, from about 99:1 to about 20:1, from about 98:1 to about 20:1, from about 95:1 to about 20:1, from about 90:1 to about 20:1, from about 80:1 to about 20:1, from about 70:1 to about 20:1, from about 60:1 to about 20:1, from about 50:1 to about 20:1, from about 40:1 to about 20:1, from about 30:1 to about 20:1, from about 22:1 to about 20:1, about 300:1, about 200:1, about 99:1, about 98.5:1, about 50:1, about 20:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, or any combination thereof.

A sixth embodiment includes the composition according to any one of the first to the fifth embodiments, wherein the at least one safener is cloquintocet-mexyl, wherein the weight ratio of the at least one phenoxy herbicide, or an agriculturally acceptable salts, esters, and amides thereof to cloquintocet-mexyl given in units of gae/ha (grams acid equivalent per hectare) to gai/ha (grams active ingredient per hectare) or gae/ha to gae/ha comprises from about 500:1 to about 1:1, from about 400:1 to about 1:1, from about 300:1 to about 1:1, from about 200:1 to about 1:1, from about 100:1 to about 1:1, from about 99:1 to about 1:1, from about 98:1 to about 1:1, from about 95:1 to about 1:1, from about 90:1 to about 1:1, from about 80:1 to about 1:1, from about 70:1 to about 1:1, from about 60:1 to about 1:1, from about 50:1 to about 1:1, from about 40:1 to about 1:1, from about 30:1 to about 1:1, from about 20:1 to about 1:1, from about 11:1 to about 1:1, from about 10:1 to about 1:1, from about 9:1 to about 1:1, from about 8:1 to about 1:1, from about 7:1 to about 1:1, from about 6:1 to about 1:1, from about 5:1 to about 1:1, from about 4:1 to about 1:1, from about 3:1 to about 1:1, from about 2:1 to about 1:1, from about 1:1 to about 1:1, about 300:1, about 200:1, about 99:1, about 98.5:1, about 50:1, about 22:1, about 20:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, or any combination thereof.

A seventh embodiment includes the composition according to any one of the first to the sixth embodiments, wherein the at least one safener is cloquintocet-mexyl, wherein the weight ratio of the at least one phenoxy herbicide, or an agriculturally acceptable salts, esters, and amides thereof to cloquintocet-mexyl given in units of gae/ha (grams acid equivalent per hectare) to gai/ha (grams active ingredient per hectare) or gae/ha to gae/ha comprises from about 500:1 to about 10:1, from about 400:1 to about 10:1, from about 300:1 to about 10:1, from about 200:1 to about 10:1, from about 100:1 to about 10:1, from about 99:1 to about 10:1, from about 98:1 to about 10:1, from about 95:1 to about 10:1, from about 90:1 to about 10:1, from about 80:1 to about 10:1, from about 70:1 to about 10:1, from about 60:1 to about 10:1, from about 50:1 to about 10:1, from about 40:1 to about 10:1, from about 30:1 to about 10:1, from about 20:1 to about 10:1, from about 11:1 to about 10:1, about 500:1 to about 20:1, from about 400:1 to about 20:1, from about 300:1 to about 20:1, from about 200:1 to about 20:1, from about 100:1 to about 20:1, from about 99:1 to about 20:1, from about 98:1 to about 20:1, from about 95:1 to about 20:1, from about 90:1 to about 20:1, from about 80:1 to about 20:1, from about 70:1 to about 20:1, from about 60:1 to about 20:1, from about 50:1 to about 20:1, from about 40:1 to about 20:1, from about 30:1 to about 20:1, from about 22:1 to about 20:1, about 300:1, about 200:1, about 99:1, about 98.5:1, about 50:1, about 20:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, or any combination thereof.

An eighth embodiment includes the composition according to any one of the first to the seventh embodiments, wherein the composition comprises the (a) at least one phenoxy herbicide, or an agriculturally acceptable salts, esters, and amides thereof comprises 2,4-D, 2,4-D EHE, 2,4-D DMA, 2,4-D choline, and/or 2,4-DB, and the (b) at least one safener comprises cloquintocet-mexyl.

A ninth embodiment includes the composition according to any one of the first to the eighth embodiments, wherein the composition comprises the (a) at least one phenoxy herbicide, or an agriculturally acceptable salts, esters, and amides thereof comprises 2,4-D EHE, 2,4-D DMA, and/or 2,4-D choline and the (b) at least one safener comprises cloquintocet-mexyl.

A tenth embodiment includes the composition according to any one of the first to the ninth embodiments, the composition further comprises at least one additional herbicide.

An eleventh embodiment includes the composition according to the tenth embodiment, wherein the at least one additional herbicide comprises fluroxypyr, fluroxypyr-meptyl, halauxifen, halauxifen-methyl, and/or pyroxsulam, or an agriculturally acceptable salts, esters, and amides thereof.

A twelfth embodiment includes the composition according to any one of the first to the eleventh embodiments, wherein the composition further comprises at least one an agriculturally acceptable agent comprising an adjuvant, and/or a carrier.

An thirteenth embodiment includes the composition according to any one of the first to the twelfth embodiments, the composition is applied to a cereal crop prior to or at the three-leaf stage of the cereal crop.

An fourteenth embodiment includes a method of protecting a cereal crop from harmful effects of a phenoxy herbicide, or an agriculturally acceptable salts, esters, and amides thereof comprising the step of contacting the cereal crop with, or applying to the area under cultivation, a herbicidally effective amount of at least one composition according to any one of the first to the thirteenth embodiments prior to the one-leaf, two-leaf, and/or three-leaf stage of the cereal crop.

A fifteenth embodiment includes the method according to the fourteenth embodiment, wherein the at least one composition according to any one of the first to the thirteenth embodiments is applied prior to or at the three-leaf stage.

A sixteenth embodiment includes the method according to any one of the fourteenth to the fifteenth embodiments, wherein the harmful effects of a phenoxy herbicide comprises growth inhibition, leaf deformity, epinasty, chlorosis, and/or delay in maturity.

A seventeenth embodiment includes a method of protecting a cereal crop from harmful effects of a phenoxy herbicide, or an agriculturally acceptable salts, esters, and amides thereof comprising the step of contacting the cereal crop with, or applying to the area under cultivation, a safener selected from the quinolinyloxyacetate family of chemicals prior to the one-leaf, two-leaf, and/or three-leaf stage, wherein the weight ratio of the phenoxy herbicide, or an agriculturally acceptable salts, esters, and amides thereof to the safener is from about 500:1 to 1:1.

An eighteenth embodiment includes the method according to the seventeenth embodiment, wherein the at least one composition according to any one of the first to the thirteenth embodiments is applied prior to or at the three-leaf stage.

A nineteenth embodiment includes the method according to any one of the seventeenth to the eighteenth embodiments, wherein the harmful effects of a phenoxy herbicide comprises growth inhibition, leaf deformity, epinasty, chlorosis, and/or delay in maturity.

A twentieth embodiment includes the method according to any one of the fourteenth to the nineteenth embodiments, wherein the amount of the composition is applied at a rate, expressed in gae/ha of a phenoxy herbicide, or an agriculturally acceptable salts, esters, and amides thereof comprises about: 100, 200, 300, 350, 400, 450, 500, 550, 600, 650, 700, 750, 800, 900, 1000, 1100, 1200, 1300, 1400, and 1500 gae/ha, or any combination thereof.

A twenty first embodiment includes the method according to the twentieth embodiment, wherein the amount of the composition is applied at a rate, expressed in gai/ha or gae/ha of a safener selected from the quinolinyloxyacetate family of chemicals, or an agriculturally acceptable salts, esters, and amides thereof comprises about: 5, 10, 20, 25, 30, 35, 40, 45, 50, 55, 60, 65, 70, 75, 80, 85, 90, 95, 100, 150, and 200 gae/ha, or any combination thereof.

A twenty second embodiment includes the method according to any one of the fourteenth to the twenty first embodiments, wherein the composition according to any one of the first to the thirteenth embodiments are applied to water.

A twenty third embodiment includes the method according to any one of the fourteenth to the twenty second embodiments, wherein the composition according to any one of the first to the thirteenth embodiments are applied pre-emergently to a cereal crop or a growing area.

A twenty fourth embodiment includes the method according to any one of the fourteenth to the twenty third embodiments, wherein the composition according to any one of the first to the thirteenth embodiments are applied post-emergently to a cereal crop or a growing area.

A twenty fifth embodiment includes a method of protecting a cereal crop from harmful effects of a phenoxy herbicide, or an agriculturally acceptable salts, esters, and amides thereof comprising the step of contacting the cereal crop with, or applying to the area under cultivation, at least one agent comprising at least one safener selected from the quinolinyloxyacetate family of chemicals prior to the one-leaf, two-leaf, and/or three-leaf stage, wherein the weight ratio of the phenoxy herbicide, or an agriculturally acceptable salts, esters, and amides thereof to the safener is from about 500:1 to 1:1.

A twenty sixth embodiment includes the method according to the twenty fifth embodiment, wherein the at least one safener is applied prior to or at the three-leaf stage.

A twenty seventh embodiment includes the method according to any one of the twenty fifth to the twenty sixth embodiments, wherein the phenoxy herbicide comprises 2,4-D, 2,4-D EHE, 2,4-D DMA, 2,4-D choline, and/or 2,4-DB.

A twenty eighth embodiment includes the method according to any one of the twenty fifth to the twenty seventh embodiments, wherein the at least one safener comprises cloquintocet acid and/or cloquintocet-mexyl.

A twenty ninth embodiment includes the method according to any one of the twenty fifth to the twenty eighth embodiments, wherein the at least one safener is cloquintocet-mexyl.

A thirtieth embodiment includes the method according to any one of the twenty fifth to the twenty ninth embodiments, wherein the at least one agent comprises at least one herbicide comprising fluroxypyr, fluroxypyr-meptyl, halauxifen, halauxifen-methyl and/or pyroxsulam, or an agriculturally acceptable salts, esters, and amides thereof.

A thirty first embodiment includes the method according to any one of the twenty fifth to the thirtieth embodiments, wherein the at least one agent comprises halauxifen-methyl, pyroxsulam, and cloquintocet acid or wherein the at least one agent comprises halauxifen-methyl, pyroxsulam, and cloquintocet mexyl.

A thirty second embodiment includes the method according to any one of the twenty fifth to the thirtieth embodiments, wherein the at least one agent comprises halauxifen-methyl and cloquintocet acid or wherein the at least one agent comprises halauxifen-methyl and cloquintocet mexyl.

A thirty third embodiment includes the method according to any one of the twenty fifth to the thirtieth embodiments, wherein the at least one agent comprises pyroxsulam and cloquintocet acid or wherein the at least one agent comprises pyroxsulam and cloquintocet mexyl.

A thirty fourth embodiment includes the method according to any one of fourteenth to the thirty third embodiments, wherein the cereal crop comprises wheat and/or barley.

A thirty fifth embodiment includes the method according to any one of fourteenth to the thirty fourth embodiments, wherein the method protects wheat from harmful effects.

A thirty sixth embodiment includes the method according to any one of the fourteenth to the thirty fourth embodiments, the method protects barley from harmful effects.

A thirty seventh embodiment includes the composition according to any one of the first to the thirteenth embodiments, wherein the composition comprises halauxifen-methyl, pyroxsulam, cloquintocet acid, and a phenoxy herbicide comprising 2,4-D, 2,4-D EHE, 2,4-D DMA, 2,4-D choline, and/or 2,4-DB.

A thirty eighth embodiment includes the composition according to any one of the first to the thirteenth embodiments, wherein the composition comprises halauxifen-methyl, cloquintocet mexyl, and a phenoxy herbicide comprising 2,4-D, 2,4-D EHE, 2,4-D DMA, 2,4-D choline, and/or 2,4-DB.

A thirty ninth embodiment includes the composition according to any one of the first to the thirteenth embodiments, wherein the composition comprises pyroxsulam, cloquintocet acid, and a phenoxy herbicide comprising 2,4-D, 2,4-D EHE, 2,4-D DMA, 2,4-D choline, and/or 2,4-DB.

A fortieth embodiment includes the composition according to any one of the first to the thirteenth and the thirty seventh to the thirty ninth embodiments, wherein the composition is applied post-emergently to wheat or a growing area.

A forty first embodiment includes the composition according to any one of the first to the thirteenth and the thirty seventh to the thirty ninth embodiments, wherein the composition is applied post-emergently to barley or a growing area.

A forty second embodiment includes any one of the first to the forty first embodiments, wherein the cereal crop comprises wheat and/or barley.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

### Definitions

Unless defined otherwise, the scientific and technology nomenclatures have the same meaning as commonly understood by a person in the ordinary skill in the art pertaining to this disclosure.

As used herein, unless explicitly stated otherwise or clearly implied otherwise the term ‘about’ refers to a range of values plus or minus 10 percent, e.g. about 1.0 encompasses values from 0.9 to 1.1.

As used herein, 2,4-D is 2-(2,4-dichlorophenoxy)acetic acid and possesses the following structure:

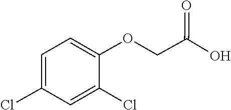 [SMILES: O=C(O)COc1ccc(Cl)cc1Cl]

Exemplary uses of 2,4-D are described in Tomlin, C., ed. A World Compendium The Pesticide Manual. 15th ed. Alton: BCPC Publications, 2009 (hereafter “The Pesticide Manual, Fifteenth Edition, 2009.”). Exemplary uses of 2,4-D include its use for post-emergence control of annual and perennial broadleaf weeds, e.g., in cereals, maize, sorghum, grassland, established turf, grass seed crops, orchards, cranberries, asparagus, sugar cane, rice forestry and non-crop land. Exemplary chemical forms of 2,4-D include salt or ester forms, for example, 2,4-D EHE, which is 2-ethylhexyl 2-(2,4-dichlorophenoxy)acetate and possesses the following structure:

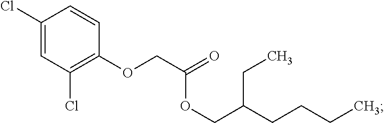 [SMILES: CCCCC(CC)COC(=O)COc1ccc(Cl)cc1Cl]

2,4-D DMA, which is 2-(2,4-dichlorophenoxy)acetic acid with N-methylmethanamine and possesses the following structure:

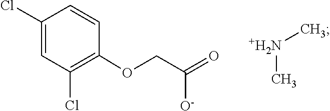

2,4-D choline, which is 2-hydroxy-N,N,N-trimethylethanaminium 2-(2,4-dichlorophenoxy)acetate and possesses the following structure:

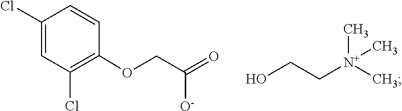

and

2,4-DB, which is 2-(2,4-dichlorophenoxy)butyric acid and possesses the following structure:

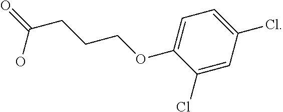 [SMILES: O=C(O)CCCOc1ccc(Cl)cc1Cl]

As used herein, halauxifen-methyl (methyl 4-amino-3-chloro-6-(4-chloro-2-fluoro-3-methoxyphenyl)picolinate) possesses the following structure:

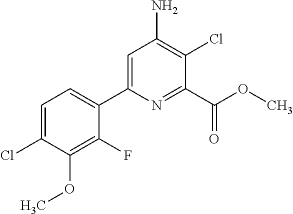 [SMILES: COC(=O)c1nc(-c2ccc(Cl)c(OC)c2F)cc(N)c1Cl]

It is described in in U.S. Pat. No. 7,314,849 B2, which is incorporated herein by reference in its entirety. Exemplary uses of halauxifen-methyl include its use to control broadleaf weeds, e.g., in cereal crops. Halauxifen-methyl may be used as other forms, e.g., halauxifen K+ (potassium 4-amino-3-chloro-6-(4-chloro-2-fluoro-3-methoxyphenyl) picolinate), which possesses the following structure:

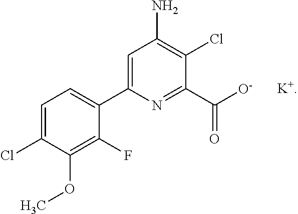 [SMILES: COc1c(Cl)ccc(-c2cc(N)c(Cl)c(C(=O)[O-])n2)c1F]

As used herein, halauxifen can include 4-Amino-3-chloro-6-(4-chloro-2-fluoro-3-methoxyphenyl)pyridine-2-carboxylic acid and 6-amino-5-chloro-2-(4-chloro-2-fluoro-3-methoxyphenyl)-pyrimidine-4-carboxylic acid derivatives. Exemplary uses of halauxifen include controlling undesirable vegetation, including but not limited to grass, broadleaf and sedge weeds, in multiple non-crop and cropping situations.

As used herein, fluroxypyr is 2-[(4-amino-3,5-dichloro-6-fluoro-2-pyridinyl)oxy]acetic acid and possesses the following structure:

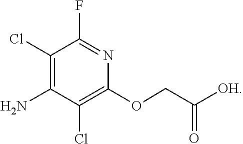 [SMILES: Nc1c(Cl)c(F)nc(OCC(=O)O)c1Cl]

Exemplary uses of fluroxypyr is described in The Pesticide Manual, Fifteenth Edition, 2009. Exemplary uses of fluroxypyr include its use for post-emergence foliar application to control broadleaf weeds, e.g., in small grain crops, control Rumex spp. and Urtilca dioica in pastures, and control Trifolium repens in amenity grassland. Other exemplary uses include its use to control herbaceous and woody broadleaf weeds, e.g., in orchards and plantation crops, and broadleaf brush, e.g., in conifer forests. Exemplary chemical forms of fluroxypyr include, for example, fluroxypyr methylheptyl ester (also known as fluroxypyr meptyl), which is 1-methylheptyl 2-[(4-amino-3,5-dichloro-6-fluoro-2-pyridinyl)oxy]acetate and possesses the following structure:

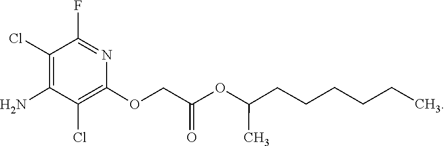 [SMILES: CCCCCCC(C)OC(=O)COc1nc(F)c(Cl)c(N)c1Cl]

As used herein, herbicide means a compound, e.g., an active ingredient that kills, controls or otherwise adversely modifies the growth of plants.

As used herein, pyroxsulam is N-(5,7-dimethoxy[1,2,4]triazolo[1,5-a]pyrimidin-2-yl)-2-methoxy-4-(trifluoromethyl)-3-pyridinesulfonamide and possesses the following structure:

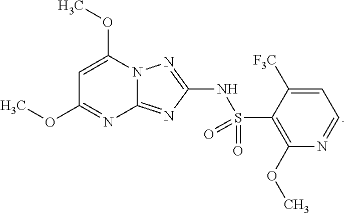 [SMILES: COc1cc(OC)n2nc(NS(=O)(=O)c3c(C(F)(F)F)ccnc3OC)nc2n1]

Its herbicidal activity is exemplified in The Pesticide Manual, Fifteenth Edition, 2009. Exemplary uses of pyroxsulam include its use as an herbicide for broad spectrum post-emergence control of annual grass and broadleaf weeds in cereals.

Safeners from the quinolinyloxyacetate family of chemicals are described in U.S. Pat. No. 4,902,340. The preferred safeners from the quinolinyloxyacetate family of chemicals are derivatives of cloquintocet, most preferably the mexyl ester. Cloquintocet is the common name for [(5-chloro-8-quinolinyl)oxy]acetic acid. Its safening activity is described in The Pesticide Manual, Fourteenth Edition, 2006. Cloquintocet is used as a safener in small grain cereals.

As used herein, a herbicidally effective or vegetation controlling amount is an amount of active ingredient which causes an adversely modifying effect to the vegetation e.g., causing deviations from natural development, killing, effecting regulation, causing desiccation, causing retardation, and the like.

As used herein, plants and vegetation include, but are not limited to, germinant seeds, emerging seedlings, plants emerging from vegetative propagules, immature vegetation, and established vegetation.

As used herein, agriculturally acceptable salts and esters refer to salts and esters that exhibit herbicidal activity, or that are or can be converted in plants, water, or soil to the referenced herbicide. Exemplary agriculturally acceptable esters are those that are or can be hydrolyzed, oxidized, metabolized, or otherwise converted, e.g., in plants, water, or soil, to the corresponding carboxylic acid which, depending on the pH, may be in the dissociated or undissociated form.

Exemplary salts include those derived from alkali or alkaline earth metals and those derived from ammonia and amines. Exemplary cations include sodium, potassium, magnesium, and ammonium cations of the formula:

R¹R²R³R⁴N⁺

wherein R1, R2, R3 and R4 each, independently represents hydrogen or C1-C12 alkyl, C3-C12 alkenyl or C3-C12 alkynyl, each of which is optionally substituted by one or more hydroxy, C1-C4 alkoxy, C1-C4 alkylthio or phenyl groups, provided that R1, R2, R3 and R4 are sterically compatible. Additionally, any two of R1, R2, R3 and R4 together may represent an aliphatic difunctional moiety containing one to twelve carbon atoms and up to two oxygen or sulfur atoms. Salts can be prepared by treatment with a metal hydroxide, such as sodium hydroxide, with an amine, such as ammonia, trimethylamine, diethanolamine, 2-methylthiopropylamine, bisallylamine, 2-butoxyethylamine, morpholine, cyclododecylamine, or benzylamine or with a tetraalkylammonium hydroxide, such as tetramethylammonium hydroxide or choline hydroxide.

Exemplary esters include those derived from C1-C12 alkyl, C3-C12 alkenyl, C3-C12 alkynyl or C7-C10 aryl-substituted alkyl alcohols, such as methyl alcohol, isopropyl alcohol, 1-butanol, 2-ethylhexanol, butoxyethanol, methoxypropanol, allyl alcohol, propargyl alcohol, cyclohexanol or unsubstituted or substituted benzyl alcohols. Benzyl alcohols may be substituted with from 1-3 substituents independently selected from halogen, C1-C4 alkyl or C1-C4 alkoxy. Esters can be prepared by coupling of the acids with the alcohol using any number of suitable activating agents such as those used for peptide couplings such as dicyclohexylcarbodiimide (DCC) or carbonyl diimidazole (CDI); by reacting the acids with alkylating agents such as alkylhalides or alkylsulfonates in the presence of a base such as triethylamine or lithium carbonate; by reacting the corresponding acid chloride of an acid with an appropriate alcohol; by reacting the corresponding acid with an appropriate alcohol in the presence of an acid catalyst or by transesterification.

### Compositions and Methods

Provided herein are safening compositions comprising an herbicidally effective amount of (a) at least one phenoxy herbicide, or an agriculturally acceptable salts, esters, and amides thereof; (b) at least one safener from quinolinyloxyacetate family of chemicals, or agriculturally acceptable salts, esters and amides thereof, wherein the weight ratio of the at least one phenoxy herbicide to cloquintocet is from about 500:1 to about 1:1.

The instant disclosure provides that the use of cloquintocet acid and/or cloquintocet-mexyl at low rates in composition with a phenoxy herbicide exhibits a protecting effect against the phytotoxicity of the phenoxy herbicide on spring and winter wheat (Triticum aestivum L; TRZAS, TRZAW), durum wheat (Triticum durum L; TRZDU) and spring and winter barley (Hordeum vulgare L; HORVS, HORVW) at herbicide-to-safener ratios between about 500:1 and about 1:1 without losing the herbicidal effects on weeds such as cleavers (Galium aparine L; GALAP), purple deadnettle (Lamium purpureum L; LAMPU), kochia (Kochia scoparia L; KCHSC), scented mayweed (Matricaria chamomila L; MATCH), com poppy (Papaver rhoeas L; PAPRH), wild buckwheat (Polygonum convolvulus L; POLCO), Russian thistle (Salsola iberica L; SASKR), chickweed (Stellaria media L; STEME), bird's-eye speedwell (Veronica persica L; VERPE), wild pansy (Viola tricolor L; VIOTR), volunteer canola (Brassica napus), shepherd's-purse (Capsella bursa-pastoris), lamb's-quarters (Chenopodium album), flixweed (Descurainia Sophia), hemp-nettle (Galeopsis tetrahit), smartweed (Polygonum persicaria), wild mustard (Sinapis arvensis), annual sowthistle (Sonchus oleraceus), dandelion (Taraxacum officinale), and stinkweed (Thlaspi arvense).

The term herbicide is used herein to mean an active ingredient that kills, controls or otherwise adversely modifies the growth of plants. An herbicidally effective or vegetation controlling amount is an amount of active ingredient which causes an adversely modifying effect and includes deviations from natural development, killing, regulation, desiccation, retardation, and the like. The terms plants and vegetation include germinant seeds, emerging seedlings and established vegetation. The term safener, as used herein, refers to a compound that selectively protects crop plants from herbicide damage without significantly reducing activity in target weed species.

Herbicidal activity is exhibited by the compounds when they are applied directly to the plant or to the locus of the plant via foliar, soil, or water application at any stage of growth or before planting or emergence. The effect observed depends upon the plant species to be controlled, the stage of growth of the plant, the application parameters of dilution and spray drop size, the particle size of solid components, the environmental conditions at the time of use, the specific compound employed, the specific adjuvants and carriers employed, the soil type, and the like, as well as the amount of chemical applied. These and other factors can be adjusted as is known in the art to promote non-selective or selective herbicidal action. Generally, it is preferred to apply the composition of the present disclosure postemergence to relatively immature undesirable vegetation to achieve the maximum control of weeds.

Cultivated plants which are to be protected from the adverse effect of undesirable plant growth may be damaged to a certain degree when an effective dose of herbicides is used. Safening means preventing the adverse effect of an herbicide on the cultivated plants, i.e., protecting the cultivated plants without, at the same time, noticeably influencing the herbicidal action on weeds to be combated.

In certain embodiments of the compositions and methods described herein, the phenoxy herbicide, i.e., 2,4-D, or agriculturally acceptable salts, esters, amides, and/or choline thereof, is employed. In certain embodiments, an ester of 2,4-D is employed. In certain embodiments, 2,4-D, 2,4-D EHE, 2,4-D DMA, 2,4-D choline, and/or 2,4-DB is employed. In certain embodiments, 2,4-D EHE, 2,4-D DMA, and/or 2,4-D choline is employed.

In certain embodiments of the compositions and methods described herein, the weight ratio of the phenoxy herbicide to the safener at which the herbicidal effect on the cultivated plant is prevented lies within the range of between about 500:1 and about 1:1. Preferably, the weight ratio of the phenoxy herbicide to the safener at which the herbicidal effect on the cultivated plant is prevented lies within the range of between about 500:1 and about 10:1, about 300:1 and about 10:1, and/or about 200:1 and about 10:1.

The rate at which the safened composition is applied will depend upon the particular type of weed to be controlled, the degree of control required, and the timing and method of application. In general, the composition disclosed herein can be applied prior to the one-leaf, two-leaf, and/or three-leaf stage of a cereal crop including, but is not limited to, wheat and barley or at an application rate from about 1 gae/ha to about 1500 gae/ha based on the total amount of phenoxy herbicide and safener in the composition. In a preferred embodiment, cloquintocet acid or cloquintocet-mexyl is applied at a rate from about 5 gae/ha to about 100 gae/ha and the phenoxy herbicide component is applied at a rate from about 100 gae/ha to about 1500 gae/ha prior to the one-leaf, two-leaf, and/or three-leaf stage.

The phenoxy herbicide and the safener disclosed herein can be applied either separately or together as part of a multipart herbicidal system.

The herbicide-safener composition disclosed herein can be applied in conjunction with one or more other herbicides to control a wider variety of undesirable vegetation. When used in conjunction with other herbicides, the composition can be formulated with the other herbicide or herbicides, tank mixed with the other herbicide or herbicides or applied sequentially with the other herbicide or herbicides. Some of the herbicides that can be employed in conjunction with the safened composition disclosed herein include: acetochlor, acifluorfen, aclonifen, AE0172747, alachlor, amidosulfuron, aminocyclopyrachlor, aminotriazole, ammonium thiocyanate, anilifos, atrazine, AVH 301, azimsulfuron, benfuresate, bensulfuron-methyl, bentazone, benthiocarb, benzobicyclon, bifenox, bispyribac-sodium, bromacil, bromoxynil, butachlor, butafenacil, butralin, cafenstrole, carbetamide, carfentrazone-ethyl, chlorflurenol, chlorimuron, chlorpropham, cinosulfuron, clethodim, clomazone, clopyralid, cloransulam-methyl, cyclosulfamuron, cycloxydim, cyhalofop-butyl, dicamba, dichlobenil, dichlorprop-P, diclosulam, diflufenican, diflufenzopyr, dimethenamid, dimethenamid-p, diquat, dithiopyr, diuron, EK2612, EPTC, esprocarb, ET-751, ethoxysulfuron, ethbenzanid, F7967, fenoxaprop, fenoxaprop-ethyl, fenoxaprop-ethyl+isoxadifen-ethyl, fentrazamide, flazasulfuron, florasulam, fluazifop, fluazifop-P-butyl, flucetosulfuron, flufenacet, flufenpyr-ethyl, flumetsulam, flumicloracpentyl, flumioxazin, fluometuron, flupyrsulfuron, fluroxypyr, fomesafen, foramsulfuron, fumiclorac, glufosinate, glufosinate-ammonium, glyphosate, halauxifen, halauxifen-methyl, halosulfuron, haloxyfop-methyl, haloxyfop-R, imazamethabenz, imazamox, imazapic, imazapyr, imazaquin, imazethapyr, imazosulfuron, indanofan, iodosulfuron, ioxynil, IR 5790, isoproturon, isoxaben, isoxaflutole, KUH-021, lactofen, linuron, MCPA, MCPA ester & amine, mecoprop-P, mefenacet, mesosulfuron, mesotrione, metamifop, metolachlor, metosulam, metribuzin, metsulfuron, molinate, MSMA, napropamide, nicosulfuron, norflurazon, OK-9701, orthosulfamuron, oryzalin, oxadiargyl, oxadiazon, oxazichlomefone, oxyfluorfen, paraquat, pendimethalin, penoxsulam, pentoxazone, pethoxamid, picloram, picolinafen, piperophos, pretilachlor, profoxydim, propachlor, propanil, propyzamide, prosulfocarb, prosulfuron, pyraclonil, pyrasulfotole, pyrazogyl, pyrazosulfuron, pyribenzoxim, pyriftalid, pyriminobac-methyl, primisulfuron, pyroxsulam, quinclorac, quizalofop-ethyl-D, S-3252, saflufenacil, sethoxydim, simazine, SL-0401, SL-0402, s-metolachlor, sulcotrione, sulfentrazone, sulfosate, tebuthiuron, terbacil, TH-547, thiazopyr, thiobencarb, triclopyr, triclopyr esters and amine, trifluralin and tritosulfuron.

The safened composition disclosed herein can, further, be used in conjunction with glyphosate, glufosinate, dicamba, imidazolinones or on glyphosate-tolerant, glufosinate-tolerant, dicamba-tolerant, or imidazolinone-tolerant crops. It is generally preferred to use the herbicide-safener composition disclosed herein in combination with herbicides that are selective for the crop being treated and which complement the spectrum of weeds controlled by these compounds at the application rate employed. It is further generally preferred to apply the safened composition disclosed herein and other complementary herbicides at the same time, either as a combination formulation or as a tank mix.

In practice, it is preferable to use the safened composition disclosed herein in mixtures containing an herbicidally effective amount of the herbicidal components along with at least one agriculturally acceptable adjuvant or carrier. Suitable adjuvants or carriers should not be phytotoxic to valuable crops, particularly at the concentrations employed in applying the compositions for selective weed control in the presence of crops and should not react chemically with herbicidal components or other composition ingredients. Such mixtures can be designed for application directly to weeds or their locus or can be concentrates or formulations that are normally diluted with additional carriers and adjuvants before application. They can be solids, such as, for example, dusts, granules, water dispersible granules, or wettable powders, or liquids, such as, for example, emulsifiable concentrates, solutions, emulsions or suspensions.

Suitable agricultural adjuvants and carriers that are useful in preparing the herbicidal compositions disclosed herein are well known to those skilled in the art. Some of these adjuvants include, but are not limited to, crop oil concentrate (mineral oil (85%)+emulsifiers (15%)); nonylphenol ethoxylate; benzylcocoalkyldimethyl quaternary ammonium salt; blend of petroleum hydrocarbon, alkyl esters, organic acid, non-ionic and anionic surfactant; C9-C11 alkylpolyglycoside; phosphated alcohol ethoxylate; natural primary alcohol (C12-C16) ethoxylate; di-sec-butylphenol EO-PO block copolymer; polysiloxane-methyl cap; nonylphenol ethoxylate+urea ammonium nitrate; emulsified methylated seed oil; tridecyl alcohol (synthetic) ethoxylate (8EO); tallow amine ethoxylate (15 EO); PEG (400) dioleate-99.

Liquid carriers that can be employed include water, toluene, xylene, petroleum naphtha, crop oil, acetone, methyl ethyl ketone, cyclohexanone, trichloroethylene, perchloroethylene, ethyl acetate, amyl acetate, butyl acetate, propylene glycol monomethyl ether and diethylene glycol monomethyl ether, methyl alcohol, ethyl alcohol, isopropyl alcohol, amyl alcohol, ethylene glycol, propylene glycol, glycerine, N-methyl-2-pyrrolidinone, N,N-dimethyl alkylamides, dimethyl sulfoxide, liquid fertilizers and the like. Water is generally the carrier of choice for the dilution of concentrates.

Suitable solid carriers include talc, pyrophyllite clay, silica, attapulgus clay, kaolin clay, kieselguhr, chalk, diatomaceous earth, lime, calcium carbonate, bentonite clay, Fuller's earth, cotton seed hulls, wheat flour, soybean flour, pumice, wood flour, walnut shell flour, lignin, and the like.

It is usually desirable to incorporate one or more surface-active agents into the compositions disclosed herein. Such surface-active agents are advantageously employed in both solid and liquid compositions, especially those designed to be diluted with carrier before application. The surface-active agents can be anionic, cationic or nonionic in character and can be employed as emulsifying agents, wetting agents, suspending agents, or for other purposes. Surfactants conventionally used in the art of formulation and which may also be used in the present formulations are described, inter alia, in “McCutcheon's Detergents and Emulsifiers Annual,” MC Publishing Corp., Ridgewood, N.J., 1998 and in “Encyclopedia of Surfactants,” Vol. Chemical Publishing Co., New York, 1980-81. Typical surface-active agents include salts of alkyl sulfates, such as diethanolammonium lauryl sulfate; alkylarylsulfonate salts, such as calciumdodecylbenzenesulfonate; alkylphenol-alkylene oxide addition products, such as nonylphenol-C18 ethoxylate; alcohol-alkylene oxide addition products, such as tridecyl alcohol-C16 ethoxylate; soaps, such as sodium stearate; alkylnaphthalene-sulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; salts of mono and dialkyl phosphate esters; vegetable oils such as soybean oil, rapeseed oil, olive oil, castor oil, sunflower seed oil, coconut oil, corn oil, cotton seed oil, linseed oil, palm oil, peanut oil, safflower oil, sesame oil, tung oil and the like; and esters of the above vegetable oils.

Other additives commonly used in agricultural compositions include compatibilizing agents, antifoam agents, sequestering agents, neutralizing agents and buffers, corrosion inhibitors, dyes, odorants, spreading agents, penetration aids, sticking agents, dispersing agents, thickening agents, freezing point depressants, antimicrobial agents, and the like. The compositions may also contain other compatible components, for example, other herbicides, plant growth regulants, fungicides, insecticides, and the like and can be formulated with liquid fertilizers or solid, particulate fertilizer carriers such as ammonium nitrate, urea and the like.

The concentration of the active ingredients in the herbicide-safener composition disclosed herein is generally from 0.001 to 98 percent by weight. Concentrations from 0.01 to 90 percent by weight are often employed. In compositions designed to be employed as 20 concentrates, the active ingredients are generally present in a concentration from 5 to 98 weight percent, preferably 10 to 90 weight percent. Such compositions are typically diluted with an inert carrier, such as water, before application. The diluted compositions usually applied to weeds or the locus of weeds generally contain 0.0001 to 1 weight percent active ingredient and preferably contain 0.001 to 0.05 weight percent.

The present compositions can be applied to weeds or their locus by the use of conventional ground or aerial dusters, sprayers, and granule applicators, or irrigation water, and by other conventional means known to those skilled in the art.

The described embodiments and following examples are for illustrative purposes and are not intended to limit the scope of the claims. Other modifications, uses, or combinations with respect to the compositions described herein will be apparent to a person of ordinary skill in the art without departing from the spirit and scope of the claimed subject matter.

### Examples

Results in Examples are field trial results.

2,4-D is a Group 4 herbicide (Phenoxy Carboxylic Acid class) commercialized for control of broadleaf weeds in cereals, rangeland and non-crop areas. 2,4-D is known to cause injury and reduce yield when applied to juvenile wheat plants, and therefore, applications prior to the four-leaf stage are not recommended (confer 2,4-D ester label and Cavanaugh et al. 1998). Injury from 2,4-D applied prior to the 4-leaf stage of spring wheat often manifests head deformity and abnormalities and can reduce yield. Cloquintocet is a safener used primarily to reduce injury caused by Group 1 and Group 2 graminicides to cereal host crops. Cloquintocet has been commercialized as a safener with graminicides including the Group 1 active ingredients such as pinoxaden and clodinafop-propargyl, and the Group 2 active ingredients such as flucarbazone-sodium and pyroxsulam. The present disclosure relates in part to a reduction in wheat growth inhibition and/or head deformity caused by 2,4-D when it is applied in a tank-mix combination with cloquintocet-containing products disclosed herein.

### 2016 Field Trial

One field trial was conducted in Alberta, Canada in 2016 to evaluate whether wheat injury caused by 2,4-D could be reduced by tank-mixing with a herbicide that is preformulated with cloquintocet acid. 2,4-D was applied as either an ester (660 gae/L 2,4-D EHE) or choline salt (456 gae/L 2,4-D choline) at a rate of 700 gae/ha. Cloquintocet acid was applied as a component of Rexade A (50 gae/kg halauxifen-methyl+150 gai/kg pyroxsulam+318.6 gai/kg cloquintocet acid) at a rate of 63.72 gae/ha. Some treatments were applied with Intake adjuvant at 1% v/v. Intake is a blend of a non-ionic surfactant and a paraffinic oil and is used to improve the spreading and wetting of herbicides. Herbicide treatments were made to spring wheat (Triticum aestivum) and durum wheat (Triticum durum) at the B12-B13 stage. Head deformity was evaluated at 42, 55 and 71 days after treatment (DAT).

This trial was established a weed-free tolerance trial. All herbicide treatments were applied post-emergence in the spring (e.g., Jun. 2, 2016). Herbicides were applied with a tractor-mounted sprayer using CO2 as a propellant. The sprayer delivered a uniform spray pattern that provided thorough coverage of the foliage using a 100 L/ha spray volume. Applications were made to spring wheat and durum wheat at the 2-3 leaf stage. Phytotoxicity to wheat was visually assessed as percent overall injury, compared to an untreated control plot. The overall injury assessments were based on visual ratings of growth inhibition, leaf deformity, epinasty, chlorosis, and delay in maturity. The primary injury observed with 2,4-D was head deformity, and therefore those are the only evaluations summarized below. The trial was designed as a randomized complete block with four replicates. Referring now to Table 1, 2,4-D ester (e.g., 2,4-D EHE) and 2,4-D choline at a rate of 700 gae/ha caused head deformity to spring wheat peaking at 8% and to durum wheat peaking at 4%. Rexade A at 40 gae/ha (delivering 63.72 gae/ha cloquintocet acid) caused no head deformity. The tank-mix of 2,4-D ester or 2,4-D choline at 700 gae/he plus Rexade A at 40 gae/ha caused no head deformity on durum wheat and <1% head deformity on spring wheat. The results provide that cloquintocet (or herbicides containing cloquintocet) could be mixed with 2,4-D ester or choline to reduce head deformity in spring wheat caused by early applications of 2,4-D.

TABLE 1
Treatments evaluated in a field study conducted in Alberta in 2016 to determine whether
wheat injury caused by 2,4-D could be reduced by tank-mixing with cloquintocet acid.

| | | | | % TRZAS Head | | | % TRZDU Head | | |
|---|---|---|---|---|---|---|---|---|---|
| | Herbicide | Safener | Adjuvant | Deformity | | | Deformity | | |
| | Rate | Rate | and | 42 | 55 | 71 | 42 | 55 | 71 |
| Treatment | (gae/ha) | (gae/ha) | Rate | DAT | DAT | DAT | DAT | DAT | DAT |
| Rexade A | 40 | 63.72 | Intake | 0.0 | 0.0 | 0.0 | 0.0 | 0.0 | 0.0 |
| | | | @1% v/v | | | | | | |
| 2,4-D Ester | 700 | 0 | 0 | 6.5 | 7.5 | 7.8 | 0.0 | 4.3 | 3.0 |
| Rexade A + | 40 + 700 | 63.72 | 0 | 0.8 | 0.0 | 0.0 | 0.0 | 0.0 | 0.0 |
| 2,4-D Ester | | | | | | | | | |
| 2,4-D Choline | 700 | 0 | Intake | 5.8 | 5.8 | 3.0 | 0.0 | 3.5 | 0.8 |
| | | | @1% v/v | | | | | | |
| Rexade A + | 40 + 700 | 63.72 | 0 | 1.3 | 0.0 | 0.0 | 0.0 | 0.0 | 0.0 |
| 2,4-D Choline | | | | | | | | | |
| Rexade A + | 40 + 700 | 63.72 | Intake | 0.0 | 0.8 | 0.8 | 0.0 | 0.0 | 0.0 |
| 2,4-D Choline | | | @1% v/v | | | | | | |
| Untreated | | | | 0.0 | 0.0 | 0.0 | 0.0 | 0.0 | 0.0 |

Abbreviations:
TRZAS, spring wheat;
TRZDU, durum wheat;
DAT, Days After Treatment

### 2017 Field Trials

Two weed-free tolerance field trials were conducted in Alberta and Manitoba, Canada in 2017 to evaluate whether wheat injury caused by 2,4-D ester could be reduced by tank-mixing with cloquintocet-containing products. All treatments are illustrated in Table 2, and include 2,4-D EHE at a rate of 700 gae/ha both alone and with an external non-ionic surfactant (NIS; Agral 90) at 0.25% v/v; the tank-mix of Rexade A (50 gae/kg halauxifen-methyl+150 gai/kg pyroxsulam+318.6 gae/kg cloquintocet acid) at 40 gae/ha plus 2,4-D ester at 700 gae/ha both with and without an NIS at 0.25% v/v; 2,4-D ester in a pre-formulation with fluroxypyr-meptyl (360 gae/L 2,4-D+90 gae/L fluroxypyr) at rates of between 350 and 800 gae/ha, Arylex WG (100 gae/kg halauxifen-methyl+100 gai/kg cloquintocet-mexyl) or Simplicity GoDri (215 gai/kg pyroxsulam+451.5 gae/kg cloquintocet acid). The rate of cloquintocet parent acid delivered by the tank-mix partners ranged from 3.5 to 63.7 gae/ha. All herbicide treatments were applied post-emergence in the spring (Jun. 16, 2017 to Jun. 26, 2017). Herbicides were applied with tractor- or bicycle-mounted sprayers using CO2 as a propellant. The sprayer delivered a uniform spray pattern that provided thorough coverage of the foliage using a 100 L/ha spray volume. Applications were made to spring wheat (Triticum aestivum) and durum wheat (Triticum durum) at the three-leaf stage. Phytotoxicity to wheat was visually assessed as percent overall injury, compared to an untreated control plot. The overall injury assessments were based on visual ratings of growth inhibition, leaf deformity, epinasty, chlorosis, and delay in maturity. The primary injury observed with 2,4-D was head deformity, and those are the only evaluations summarized below. Head deformity was evaluated at 10 to 12 weeks after treatment (WAT). The trial was designed as a randomized complete block with four replicates.

Referring now to Table 2, 2,4-D ester (e.g., 2,4-D EHE), either alone or in a preformulated with fluroxypyr-meptyl, at rates of 350-800 gae/ha caused head deformity to spring wheat peaking at 10% and to durum wheat peaking at 4%. Cloquintocet-containing products, Rexade A, Arylex WG, and Simplicity GoDri caused no or negligible head deformity. Tank-mixes of 2,4-D ester, alone or in a preformulated mixture with fluroxypyr-meptyl, with cloquintocet-containing products caused either no or significantly less head deformity on spring wheat and durum wheat. Table 2 indicates that injury to wheat caused by application of 2,4-D ester at the B13 growth stage can be mitigated by tank-mixing with cloquintocet-containing herbicides. Presently, it is recommended that application of 2,4-D containing mixtures to cereal crops must be delayed until at least the B14 growth stage (four-leaf stage; Stage 14 based on BBCH Growth Stages) to avoid crop injury, which decreases the length of the application window and enhances the amount of time the crop is forced to compete with weeds. The results shown in Table 2 provide that cloquintocet (or herbicides containing cloquintocet) could be mixed with 2,4-D ester, or other 2,4-D structural analogs, to reduce head deformity in spring wheat and durum wheat caused by applications made prior to tillering. Thus, the instant disclosure provides viability of a co-pack, tank-mix or co-formulation of 2,4-D with cloquintocet acid, cloquintocet-mexyl, or a cloquintocet-containing herbicide for safe application to spring wheat and durum wheat prior to or at B12-B13 growth stages based on BBCH Growth Stages (e.g., two-leaf to three-leaf stages).

TABLE 2
Treatments evaluated in field studies conducted in Alberta and Manitoba in 2017 to determine whether
wheat injury caused by 2,4-D could be reduced by tank-mixing with cloquintocet-containing products.

| | | | | % TRZAS Head | | | % TRZDU Head | | |
|---|---|---|---|---|---|---|---|---|---|
| | | | | Deformity | | | Deformity | | |
| | Herbicide | Safener | Adjuvant | Field | Field | | Field | Field | |
| | Rate | Rate | and | Trial 1 | Trial 2 | | Trial 1 | Trial 2 | |
| Treatment | (gae/ha) | (gae/ha) | Rate | 10 WAT | 10 WAT | 12 WAT | 10 WAT | 10 WAT | 12 WAT |
| 2,4-D Ester | 350 | 0 | 0 | 0.8 | 0.8 | 1.2 | 0.5 | 0.0 | 0.0 |
| Rexade A + | 20 + 350 | 31.9 | 0 | 0.8 | 0.5 | 0.2 | 0.8 | 0.0 | 0.0 |
| 2,4-D Ester | | | | | | | | | |
| Rexade A + | 20 + 350 | 31.9 | Agral 90 | 0.0 | 1.0 | 0.2 | 0.0 | 0.0 | 0.0 |
| 2,4-D Ester | | | @0.25% v/v | | | | | | |
| 2,4-D Ester | 700 | 0 | 0 | 1.0 | 3.5 | 3.0 | 1.2 | 0.0 | 0.0 |
| 2,4-D Ester | 700 | 0 | Agral 90 | 2.0 | 9.5 | 7.2 | 2.2 | 0.0 | 0.0 |
| | | | @0.25% v/v | | | | | | |
| Arylex + | 5 + 700 | 3.5 | 0 | 0.8 | 3.2 | 1.2 | 0.8 | 0.0 | 0.0 |
| 2,4-D Ester | | | | | | | | | |
| Arylex + | 10 + 700 | 7.1 | 0 | 0.2 | 2.0 | 0.2 | 0.2 | 0.0 | 0.0 |
| 2,4-D Ester | | | | | | | | | |
| Rexade A + | 40 + 700 | 63.7 | 0 | 1.2 | 1.0 | 0.2 | 1.2 | 0.0 | 0.0 |
| 2,4-D Ester | | | | | | | | | |
| Rexade A | 20 | 31.9 | Agral 90 | 0.0 | 0.2 | 0.0 | 0.0 | 0.0 | 0.0 |
| | | | @0.25% v/v | | | | | | |
| Rexade A | 40 | 63.7 | Agral 90 | 0.0 | 0.5 | 0.0 | 0.0 | 0.0 | 0.0 |
| | | | @0.25% v/v | | | | | | |
| OcTTain | 500 | 0 | Agral 90 | 2.5 | 6.2 | 1.5 | 3.2 | 0.0 | 0.0 |
| XL | | | @0.25% v/v | | | | | | |
| Simplicity | 15 + 500 | 31.9 | Agral 90 | 0.2 | 0.5 | 0.0 | 0.2 | 0.0 | 0.0 |
| GoDri + | | | @0.25% v/v | | | | | | |
| OcTTain | | | | | | | | | |
| XL | | | | | | | | | |
| OcTTain | 1000 | 0 | Agral 90 | 2.8 | 9.0 | 2.8 | 3.5 | 0.0 | 0.0 |
| XL | | | @0.25% v/v | | | | | | |
| Simplicity | 30 + 1000 | 63.7 | Agral 90 | 0.8 | 1.0 | 0.5 | 1.5 | 0.0 | 0.0 |
| GoDri + | | | @0.25% v/v | | | | | | |
| OcTTain | | | | | | | | | |
| XL | | | | | | | | | |
| Simplicity | 15 | 31.9 | Agral 90 | 1.8 | 0.8 | 0.0 | 2.0 | 0.0 | 0.0 |
| GoDri | | | @0.25% v/v | | | | | | |
| Simplicity | 30 | 63.7 | Agral 90 | 1.5 | 0.0 | 0.0 | 1.8 | 0.0 | 0.0 |
| GoDri | | | @0.25% v/v | | | | | | |
| Untreated | | | | 0.0 | 0.0 | 0.0 | 0.0 | 0.0 | 0.0 |

Abbreviations:
TRZAS, spring wheat;
TRZDU, durum wheat;
WAT, Weeks After Treatment

### 2018 Field Trials

Three field trials were conducted in Alberta, Saskatchewan and Manitoba, Canada in 2018 to evaluate whether wheat injury caused by 2,4-D ester could be reduced by tank-mixing with cloquintocet-containing products. 2,4-D ester was applied alone at a rate of 700 gae/ha (660 gae/L 2,4-D EHE) without an adjuvant, alone at 700 gae/ha with an NIS adjuvant (Agral 90) at 0.25% v/v, or applied at a rate of 700 gae/ha in tank-mixtures with Cloquintocet-acid (250 gae/kg cloquintocet acid) at rates between 5 and 40 gae/ha and an NIS adjuvant at 0.25% v/v. Herbicide treatments were made to spring wheat (Triticum aestivum) and durum wheat (Triticum durum) at the B12-13 stage. Growth inhibition was evaluated at 7, 14, and 66 days after treatment (DAT), head deformity was evaluated at 66 days after treatment (DAT), and yield was taken at 77 days after treatment (DAT). All treatments are illustrated in Table 3.

Weed-free tolerance trials were established. All herbicide treatments were applied post-emergence in the spring (May 28, 2018 to Jun. 1, 2018). Herbicides were applied with tractor- or bicycle-mounted sprayers using CO2 as a propellant. The sprayer delivered a uniform spray pattern that provided thorough coverage of the foliage using a 100 L/ha spray volume. Applications were made to spring wheat and durum wheat at the two-leaf to three-leaf stage. Phytotoxicity to wheat was visually assessed as percent overall injury, compared to an untreated control plot. The overall injury assessments were based on visual ratings of growth inhibition, leaf deformity, epinasty, chlorosis, and delay in maturity. The primary injury observed with 2,4-D was growth inhibition, head deformity, and yield loss, therefore those are the only evaluations summarized below. The trial was designed as a randomized complete block with four replicates. Referring now to Table 3, 2,4-D ester applied alone or with Agral 90 caused growth inhibition, head deformity and yield loss to spring and durum wheat. Tank-mixing cloquintocet-acid, at certain rates, with 2,4-D ester reduced growth inhibition, head deformity and yield loss to negligible levels (Table 3). This result provides that cloquintocet (or herbicides containing cloquintocet) could be mixed with 2,4-D ester, or other 2,4-D structural analogs, to reduce growth inhibition, head deformity and yield loss in spring wheat and durum wheat caused by applications made prior to tillering.

The results of the instant disclosure provide that injury to wheat caused by application of 2,4-D ester at the B12-B13 growth stage can be mitigated by tank-mixing with cloquintocet or cloquintocet-containing herbicides. Presently, it is recommended that application of 2,4-D containing mixtures to cereal crops must be delayed until at least the B14 growth stage to avoid crop injury, which decreases the length of the application window and enhances the amount of time the crop is forced to compete with weeds. The instant disclosure provides the viability of a co-pack, tank-mix or co-formulation of 2,4-D with cloquintocet acid, cloquintocet-mexyl, or a cloquintocet-containing herbicide for safe application to spring wheat and durum wheat prior to or at B12-B13 growth stages.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety to the extent they are not inconsistent with the explicit teachings of this specification.

TABLE 3
Treatments evaluated in field studies conducted in Alberta, Saskatchewan and Manitoba in 2018 to determine
whether wheat injury caused by 2,4-D could be reduced by tank-mixing with cloquintocet (CQC) products.

| | | | | Field Trial 1 | | | | Field Trial 2 | | Field Trial 3 | | | |
|---|---|---|---|---|---|---|---|---|---|---|---|---|---|
| | | | Adjuvant | % Growth | | % Head | | % Yield | | % Growth | | % Growth | |
| | Herbicide | Safener | and | Inhibition | | Deformity | | Loss | | Inhibition | | Inhibition | |
| | Rate | Rate | Rate | (66 DAT) | | (66 DAT) | | (77 DAT)* | | (7 DAT) | | (14 DAT) | |
| Treatment | (gae/ha) | (gae/ha) | (% v/v) | TRZAS | TRZDU | TRZAS | TRZDU | TRZAS | TRZDU | TRZAS | TRZDU | TRZAS | TRZDU |
| 2,4-D Ester | 700 | 0 | 0 | 10.1 | 2.6 | 40 | 5 | 4.5 | 6.1 | 4.5 | 5.5 | 7 | 7 |
| 2,4-D Ester | 700 | 0 | Agral 90 @ | 9.1 | 1.9 | 40 | 5.3 | 3.3 | 8.8 | 8 | 8.5 | 7.5 | 8.5 |
| | | | 0.25% | | | | | | | | | | |
| 2,4-D Ester + | 700 | 5 | Agral 90 @ | 2.6 | 0.1 | 28.8 | 0 | 0 | 0 | 4.5 | 4.5 | 8 | 7 |
| CQC acid | | | 0.25% | | | | | | | | | | |
| 2,4-D Ester + | 700 | 10 | Agral 90 @ | 3.6 | 0.9 | 21.3 | 1.5 | 0 | 0 | 4 | 4.5 | 6 | 7 |
| CQC acid | | | 0.25% | | | | | | | | | | |
| 2,4-D Ester + | 700 | 20 | Agral 90 @ | 1.3 | 0.1 | 14.5 | 3.3 | 1.1 | 0 | 5 | 4.5 | 6 | 6.5 |
| CQC acid | | | 0.25% | | | | | | | | | | |
| 2,4-D Ester + | 700 | 40 | Agral 90 @ | 1.3 | 1.5 | 0.8 | 0.8 | 2.1 | 3.6 | 2 | 2.5 | 0 | 4 |
| CQC acid | | | 0.25% | | | | | | | | | | |
| Untreated | | | | 1.3 | 1.5 | 0.8 | 0 | N/A | N/A | 0 | 0 | 0 | 0 |

*Relative to commercial standard, REXADE A at 20 gae/ha
Abbreviations:
TRZAS, spring wheat;
TRZDU, durum wheat;
DAT, Days After Treatment;
N/A, Not Available

What is claimed is:

1. A composition for protecting a cereal crop from harmful effects of a phenoxy herbicide, comprising at least one phenoxy herbicide and a safener from the quinolinyloxyacetate family of chemicals, or agriculturally acceptable salts, esters and amides thereof, wherein the weight ratio of the at least one phenoxy herbicide to cloquintocet is from about 500:1 to about 1:1.

2. The composition according to claim 1, wherein the at least one phenoxy herbicide, or an agriculturally acceptable salts, esters, and amides thereof comprises 2,4-D, 2,4-D EHE, 2,4-D DMA, 2,4-D choline, and/or 2,4-DB.

3. The composition according to claim 1, wherein the (b) at least one safener from quinolinyloxyacetate family of chemicals comprises cloquintocet, or an agriculturally acceptable salts, esters, and amides thereof.

4. The composition according to claim 1, wherein the (b) at least one safener comprises cloquintocet acid and/or cloquintocet-mexyl.

5. The composition according to claim 1, wherein the (b) at least one safener is cloquintocet-mexyl.

6. The composition according to claim 1, wherein the composition comprises the (a) at least one phenoxy herbicide, or an agriculturally acceptable salts, esters, and amides thereof comprises 2,4-D EHE, 2,4-D DMA, and/or 2,4-D choline.

7. The composition according to claim 1, further comprising an agriculturally acceptable adjuvant or carrier.

8. The composition according to claim 1, further comprising at least one additional herbicide safener.

9. The composition according to claim 1, further comprising at least one additional herbicide.

10. The composition according to claim 8, wherein the at least one additional herbicide comprises fluroxypyr, fluroxypyr-meptyl, halauxifen, halauxifen-methyl and/or pyroxsulam, or an agriculturally acceptable salts, esters, and amides thereof.

11. The composition according to claim 1, wherein the weight ratio between (a) and (b) is from about 200:1 to about 10:1.

12. A method of protecting a cereal crop from harmful effects of a phenoxy herbicide, comprising the step of contacting the cereal crop with, or applying to the area under cultivation, a herbicidally effective amount of at least one composition according to claim 1 prior to the one-leaf, two-leaf, and/or three-leaf stage of the cereal crop.

13. The method according to claim 12, wherein the at least one composition according to claim 1 is applied prior to or at the three-leaf stage.

14. The method according to claim 12, wherein the harmful effects of a phenoxy herbicide comprises growth inhibition, leaf deformity, epinasty, chlorosis, and/or delay in maturity.

15. A method of protecting a cereal crop from harmful effects of a phenoxy herbicide, or an agriculturally acceptable salts, esters, and amides thereof comprising the step of contacting the cereal crop with, or applying to the area under cultivation, at least one safener selected from the quinolinyloxyacetate family of chemicals prior to the one-leaf, two-leaf, and/or three-leaf stage, wherein the weight ratio of the phenoxy herbicide, or an agriculturally acceptable salts, esters, and amides thereof to the safener is from about 500:1 to 1:1.

16. The method according to claim 15, wherein the at least one safener is applied prior to or at the three-leaf stage.

17. The method according to claim 15, wherein the phenoxy herbicide comprises 2,4-D, 2,4-D EHE, 2,4-D DMA, 2,4-D choline, and/or 2,4-DB.

18. The method according to claim 15, wherein the at least one safener comprises cloquintocet acid and/or cloquintocet mexyl.

19. The method according to claim 15, wherein the at least one safener is cloquintocet mexyl.

20. A method of protecting a cereal crop from harmful effects of a phenoxy herbicide, or an agriculturally acceptable salts, esters, and amides thereof comprising the step of contacting the cereal crop with, or applying to the area under cultivation, at least one agent comprising at least one safener selected from the quinolinyloxyacetate family of chemicals prior to the one-leaf, two-leaf, and/or three-leaf stage of the cereal crop, wherein the weight ratio of the phenoxy herbicide, or an agriculturally acceptable salts, esters, and amides thereof to the safener is from about 500:1 to 1:1.

21.-32. (canceled)
